(19) United States
(12) Patent Application Publication
(10) Pub. No.: US 2012/0308660 A1
(43) Pub. Date: Dec. 6, 2012

(54) NANOCOATINGS FOR BIOLOGICAL MATERIALS

(75) Inventors: Amish PATEL, San Diego, CA (US); Paul W. SHABRAM, San Diego, CA (US)

(73) Assignee: PAXVAX, INC., San Diego, CA (US)

(21) Appl. No.: 13/486,949

(22) Filed: Jun. 1, 2012

Related U.S. Application Data
(60) Provisional application No. 61/492,597, filed on Jun. 2, 2011.

(51) Int. Cl. A61K 39/235; A61P 31/14; A61K 9/50

(52) U.S. Cl. 424/491; 424/233.1

(57) ABSTRACT

The present invention provides formulations comprising nanocoated biological materials (e.g., viral particles), methods for producing powders comprising nanocoated biological materials, and powders produced from such formulations and methods. Also provided are pharmaceutical compositions comprising the present formulations or dried powders, and vaccines comprising the present formulations or dried powders. The nanocoated biological materials typically display superior stability for either direct use in a formulation or in drying processes to produce a powder material, wherein the coated materials are typically more tolerant to environmental stress (e.g., chemical, thermal, and/or mechanical stress) during storage or drying processes.

### CROSS-REFERENCE TO RELATED APPLICATIONS

This application claims priority to U.S. Provisional Application Ser. No. 61/492,597, filed Jun. 2, 2011, which is hereby incorporated by reference in its entirety for all purposes.

### FIELD OF THE INVENTION

The present disclosure relates to nanofilm coated biological materials, methods of making the same, liquid and dry preparations thereof and related products.

### BACKGROUND OF THE INVENTION

The preservation and storage of biologically active materials (e.g., viruses, proteins, cells, etc.) is important for medical and research purposes. For example, storage of dehydrated biologically active materials carries with it significant benefits, such as reduced weight and volume relative to solutions of such materials. Furthermore, such materials can, under certain conditions, have increased stability relative to their solution-phase counterparts. However, producing such dehydrated compositions comprising sensitive biological materials that may be unstable (e.g., thermally, chemically, mechanically, etc.) can be problematic.

For example, one common problem with vaccines (e.g., liquid formulations and/or dry powders) is that they generally require cold storage conditions prior to administration. Cold storage is the generally accepted practice for storing, e.g., vaccine products. However, the need for cold storage is a significant barrier for vaccine distribution in developing nations and/or in other circumstances where cold storage may not be feasible or economical (e.g., field administration in remote areas). While efforts have been devoted to providing a solution for the preservation of biological materials (e.g., vaccines) that could eliminate the need for cold storage, such efforts have been relatively unsuccessful. Accordingly, there is a need for stable (e.g., thermally, chemically, mechanically, etc.) biological products (e.g., vaccines) as liquid formulations and/or dry powders which do not require cold storage conditions, and methods of producing such products.

### SUMMARY OF THE INVENTION

The present invention is based, at least in part, on the discovery that formulations comprising biological materials (e.g., viral particles) having a nanocoating thereon can impart stability to the biological materials (e.g., viral particles), such as chemical, thermal, and/or mechanical stability. The biological activity of the materials can be substantially preserved in such formulations and indeed, can be improved.

In some embodiments, the present invention provides for application of a first coating of a material having one charge onto a biological material (e.g., a viral particle) having an opposite charge. In some embodiments, a second coating is applied having a charge opposite of that of the first coating. Additional coatings may be applied, each having a charge opposite of the last coating applied. The coatings formed are in the nanometer scale and typically take the shape of the substrate on which they are coated (e.g., adenovirus, etc.). A single coating may be applied, or multiple alternating polycation and polyanion polymer coatings, that are typically biocompatible and can increase biological activity (e.g., uptake, infectivity). Such nanocoated biological materials can be incorporated in a formulation suitable for liquid storage, as well as used in the production of a corresponding dry product. In some embodiments, the present invention also provides methods for drying (e.g., spray-drying or freeze-drying) nanocoated biological materials (e.g., viral particles). For example, nanocoated virus particle powders which retain their viral infectivity can be produced from liquid formulations described herein. In addition, biological material comprised in formulations described herein can be stable (e.g., chemically, thermally, and/or mechanically, etc.), such as more stable than the uncoated biological material.

Accordingly, in a first aspect, the present invention relates to a formulation comprising biological materials (e.g., viral particles) having a nanocoating thereon. In some embodiments, a formulation further comprises one or more entities selected from a cryoprotectant, a surfactant, a buffer, or a combination thereof. In another aspect, the present invention relates to a method for producing a powder comprising nanocoated biological materials (e.g., viral particles), comprising drying the present formulations. In some embodiments, the formulation comprises an enteric polymer. In certain embodiments, the present invention further comprises a method for producing a powder comprising enteric polymer coated nanocoated biological materials (e.g., viral particles), comprising drying the present formulations. In some embodiments, drying the present formulations includes freeze-drying or spray-drying.

In still other aspects, the present invention relates to dried powders comprising nanocoated biological materials (e.g., viral particles) produced by the present methods and/or from the present formulations. In some embodiments, the activity of a dried, nanocoated biological material (e.g., a dried nanocoated viral particle) is at least about 70% of the activity (e.g., infectivity) prior to drying. In still other embodiments, the present invention relates to pharmaceutical compositions, such as for oral and/or parenteral administration, comprising the present dried powders comprising nanocoated biological materials (e.g., viral particles). In certain embodiments, the present invention relates to vaccines comprising the present formulations and/or dried powders. In still other embodiments, the present invention relates to a replication competent vaccine for oral or parenteral administration comprising the present formulations and/or dried powders.

### BRIEF DESCRIPTION OF THE DRAWINGS

FIG. 1 shows an exemplary process for preparing a nanocoated adenovirus.

FIG. 2 shows a graph plotted for ζ-potential as a function of number of layers of polyelectrolytes for an exemplary nanocoated viral particle. The polyelectrolyte assembly comprises of two bilayer of polyethyleneimine (PEI)- and PSS poly(styrene sulfonate) and two bilayer of polyethyleneimine (PEI)- and bovine serum albumin (BSA) on net negatively charged adenovirus (Ad4-GFP), where the naked adenovirus is represented by the left-most data point.

FIG. 3 shows the change in adenovirus particle charge upon layering of polyethyleneimine (PEI) and poly(sodium 4-styrenesulfonate) (PSS) coats.

FIG. 4 shows the change in adenovirus particle charge upon layering of a poly(allylamine hydrochloride) (PAH)-polycation coat.

FIG. 5 shows the change in adenovirus particle charge upon layering of a protamine sulfate (PS)-polycation coat.

### DETAILED DESCRIPTION

The present invention is based, at least in part, on the discovery that formulations comprising biological materials having a nanocoating thereon can impart stability to the biological materials, such as chemical, thermal, and/or mechanical stability. In some embodiments, the biological activity of the materials is substantially preserved in such formulations and indeed, can be improved. For example, in the case of viral particles, viral particles of formulations can have similar or even improved infectivities compared to, e.g., uncoated viral particles and/or viral particles not comprised in such formulations.

A variety of biological materials can be nanocoated and employed in a formulation. For example, the biological material can be a viral particle, a bacterial particle, a protein, or DNA particle, or a combination thereof.

### Viral Particles

Viral particles can be in any form, including live and attenuated as well as inactivated or killed, provided that the integrity of the antigenic determinant(s) is maintained. In some embodiments, viral particles may be useful in vaccine applications. For example, viral particles may be adenoviral particles useful for vaccines. In some embodiments, viral particles comprise adenoviral particles such as those described in co-pending U.S. patent application Ser. No. 12/847,767 (U.S. Publ. No. 2011/0123564). In some embodiments, a viral particle is a replication competent virus particle, e.g., an adenovirus that is replication competent.

In some embodiments, an adenovirus vector as described herein may be a nonenveloped icosahedral virus containing the genomic code for a specific target. Typical adenovirus structures may range from 70 to 100 nanometers in size. Adenovirus particles typically possess a net negative charge. The net surface charge may be determined using zeta potential measurement (see, e.g., Alemany et al. J. of Gen. Virol. (2000) 81:2605-2609). The adenovirus structure encodes the genomic code which is to be delivered at a target cell.

The quantity of viral particles in a formulation can, for example, be determined on the basis of their infectivity. A target or pre-determined infectivity level or activity level may be obtained by increasing or decreasing the amount of viral complexes, such as increasing or decreasing the amount present in formulation comprising the viral complexes.

The concentration or amount of adenoviral particles can be measured using anion-exchange high-performance liquid chromatography (AE-HPLC). This method has been widely adopted for adenoviral vectors used for gene therapy applications. The method utilizes an analytical anion-exchange column. Anion exchange chromatography relies on charge-to-charge interaction between the analyte and the resin in the column. Since adenovirus typically has net negative charge, it binds to the immobilized positively charged functional groups of the column resin. The bound adenovirus can then be “eluted” off the column by eluting the column with a solvent or a mixture comprising one or more components (e.g., salts) which binds to the column resin (either as a uniform concentration or via gradient elution), dislodging the bound adenovirus from the column, which can then be detected in the column eluate (e.g., by UV-vis, etc.).

### Nanocoatings

Nanocoatings described herein are charged materials that are applied to a biological material. Without being bound by theory, it is believed that the primary forces driving the coatings are based on electrostatic attractions and/or covalent bonds, but can also involve hydrogen bonding, hydrophilic forces, hydrophobic forces and/or other non-specific molecular interactions. The nanocoatings formed by polyanions and/or polycations can, in some embodiments, create strong ionic bonds that can be stable (e.g., thermally, chemically, mechanically etc.) for long periods of time under proper storage conditions (e.g., temperature, etc.). As noted above, the nanocoatings typically take the shape of the coated substrate (e.g., an adenovirus). Such nanocoatings can be characterized to determine their thickness or morphology by known microscopy techniques (e.g., SEM, etc.). The coating technique is typically performed on a substrate having an electrostatic charge (e.g., a net positive or negative charge). The coating material typically has a net charge opposite that of the substrate to be coated. A single layer of material can be deposited on the substrate, or alternating layers of positively charged and negatively charged materials can be deposited in successive layers, providing a multi-layer structure comprising a core biological material (e.g., core viral particle) and one or more layers of charged materials. For example, adenovirus vectors typically have a net negative surface charge, which makes them suitable substrates for this coating technology.

Each nanocoating typically has a thickness on the order of nanometers (e.g., about 0.01 nm, about 0.1 nm, about 1 nm, about 2 nm, about 3 nm, about 4 nm, about 5 nm, about 6 nm, about 7 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, or any other value or range of values therein).

Deposition of charged materials on an oppositely charged substrate generally takes place in seconds or minutes (e.g., about 1 second, about 5 seconds, about 10 seconds, about 15 seconds, about 20 seconds, about 30 seconds, about 40 seconds, about 50 seconds, about 1 minute, about 2 minutes, about 5 minutes, or any other value or range of values therein).

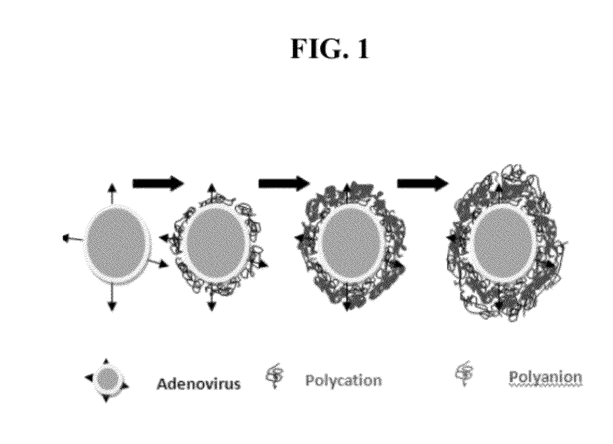

After each step of deposition of a layer, there are typically intermediate washes applied for removal of loosely attached polyionic materials. However, such washing steps can be eliminated by addition of an optimal concentration of the coating material. This can control the precision of nanocoating formation. FIG. 1 shows an exemplary process for preparation of a nanocoated adenovirus. An adenovirus core, having a net negative charge, is initially treated with a suitable polycation to form a nanocoating thereon. Then, the particle, now having a net positive charge, is treated with a polyanion to form another layer on the first polycation layer. The resultant particle now has two layers disposed thereon, and a net negative charge. Additional coating layers can be applied as desired to achieve a desired number of layers.

As described herein, nanocoatings can comprise one or more layers of an anionic or cationic material. Exemplary anionic materials include poly(sodium 4-styrenesulfonate) (PSS), hyaluronic acid (e.g., hyaluronic acid sodium salt), dextran sulfate (e.g., dextran sulfate sodium salt), bovine serum albumin (BSA), polyacrylic acid, polyaspartic acid, polyvinylsulfonate, polyacrylamidomethyl propane sulfonic acid, polylactic acid, poly(ethylene glycol-co-methacrylic acid), gelatin, alginate, chondroitin sulfate, dioleoylphosphatidylethanolamine, phosphatidylglycerol, phosphatidic acid, phosphatidylserine, and combinations thereof. The amount of anionic material can range, for example, from about 0.001 nM to about 100 mM. For example, in some embodiments, the amount ranges from about 0.01 nM to about 10 mM. In some embodiments, the amount ranges from about 0.1 nM to about 1 mM. In some embodiments, the amount ranges from about 1 nM to about 100 μM. In some embodiments, the amount ranges from about 100 nM to about 50 μM. In some embodiments, the amount ranges from about 10 nM to about 1 μM.

In some embodiments, the amount of anionic material employed (e.g., BSA) employed ranges from about 0.1 mM to about 2.0 mM. In some embodiments, the amount of anionic material (e.g., BSA) ranges from about 0.5 mM to about 1.5 mM. In some embodiments, the amount of anionic material (e.g., BSA) is about 1 mM. In some embodiments, the amount of anionic material employed (e.g., PSS) employed ranges from about 100 nM to about 300 nM. In some embodiments, the amount of anionic material (e.g., PSS) ranges from about 150 nM to about 225 nM. In some embodiments, the amount of anionic material (e.g., PSS) is about 200 nM. In some embodiments, the amount of anionic material employed (e.g., alginate) employed ranges from about 15 μM to about 35 μM. In some embodiments, the amount of anionic material (e.g., alginate) ranges from about 20 μM to about 30 μM. In some embodiments, the amount of anionic material (e.g., alginate) is about 25 μM. In some embodiments, the amount of anionic material employed (e.g., hyaluronic acid) employed ranges from about 0.1 μM to about 2 μM. In some embodiments, the amount of anionic material (e.g., hyaluronic acid) ranges from about 0.5 μM to about 1.5 μM. In some embodiments, the amount of anionic material (e.g., hyaluronic acid) is about 1 μM.

Exemplary cationic materials include poly(allylamine hydrochloride) (PAH), poly(diallyldimethylammonium chloride), poly(etheylene glycol-co-dimethylaminoethyl methacrylate) protamine sulfate (PS), hexadimethrine bromide, chitosan, poly-L-lysine hydrobromide, poly-L-arginine, polyethyleneimine (PEI), DEAE-dextran, lipofectin, lipofectamine, dioctadecylamidoglycylspermine, [N—(N9,N9-dimethylaminoethane)carbamoyl]cholesterol, 1,2-di-(9Z-octadecenoyl)-3-trimethylammonium-propane methyl sulfate salt, 1,2-ditetradecanoyl-3-dimethylammonium-propane, dimethyldioctadecylammonium bromide salt, and combinations thereof. The amount of cationic material can range, for example, from about 0.001 nM to about 100 mM. For example, in some embodiments, the amount ranges from about 0.01 nM to about 10 mM. In some embodiments, the amount ranges from about 0.1 nM to about 1 mM. In some embodiments, the amount ranges from about 1 nM to about 100 μM. In some embodiments, the amount ranges from about 100 nM to about 100 μM. In some embodiments, the amount ranges from about 10 nM to about 10 μM.

In some embodiments, the amount of cationic material employed (e.g., PEI) employed ranges from about 100 nM to about 250 nM. In some embodiments, the amount of cationic material (e.g., PEI) ranges from about 150 nM to about 210 nM. In some embodiments, the amount of cationic material (e.g., PEI) is about 180 nM. In some embodiments, the amount of cationic material employed (e.g., protamine sulfate) ranges from about 250 nM to about 400 nM. In some embodiments, the amount of cationic material (e.g., protamine sulfate) ranges from about 280 nM to about 360 nM. In some embodiments, the amount of cationic material (e.g., protamine sulfate) is about 320 nM. In some embodiments, the amount of cationic material employed (e.g., poly-L-arginine) ranges from about 200 nM to about 400 nM. In some embodiments, the amount of cationic material employed (e.g., poly-L-arginine) ranges from about 275 nM to about 325 nM. In some embodiments, the amount of cationic material employed (e.g., poly-L-arginine) is about 300 nM. In some embodiments, the amount of cationic material employed (e.g., lipofectin) ranges from about 100 nM to about 300 nM. In some embodiments, the amount of cationic material employed (e.g., lipofectin) ranges from about 175 nM to about 225 nM. In some embodiments, the amount of cationic material employed (e.g., lipofectin) is about 200 nM. In some embodiments, the amount of cationic material employed (e.g., lipofectamine) ranges from about 100 nM to about 300 nM. In some embodiments, the amount of cationic material employed (e.g., lipofectamine) ranges from about 175 nM to about 225 nM. In some embodiments, the amount of cationic material employed (e.g., lipofectamine) is about 200 nM. In some embodiments, the amount of cationic material employed (e.g., PAH) ranges from about 300 nM to about 500 nM. In some embodiments, the amount of cationic material employed (e.g., PAH) ranges from about 375 nM to about 425 nM. In some embodiments, the amount of cationic material employed (e.g., PAH) is about 400 nM. In some embodiments, the amount of cationic material employed (e.g., chitosan) ranges from about 10 μM to about 100 μM. In some embodiments, the amount of cationic material employed (e.g., chitosan) ranges from about 25 μM to about 75 μM. In some embodiments, the amount of cationic material employed (e.g., chitosan) is about 50 μM.

Further non-limiting examples of types and amounts of anionic and/or cationic materials that can be employed in the nanocoatings described herein are shown in Table 1:

TABLE 1
Exemplary Nanocoatings

| Viral Particle + Nanocoating | Concentration of Nanocoatin2 Components |
|---|---|
| Ad + PEI | PEI 180 nM |
| Ad + PEI + BSA | PEI 180 nM, BSA 1 mM |
| Ad + PEI + BSA + PEI | PEI 180 nM, BSA 1 mM, PEI 180 mM |
| Ad + PEI + BSA + PEI + BSA | PEI 180 nM, BSA 1 mM, PEI 180 mM, BSA 1 mM |
| Ad + PEI | PEI 180 nM |
| Ad + PEI + PSS | PEI 180 nM, PSS 200 nM |
| Ad + PEI + PSS + PEI | PEI 180 nM, PSS 200 nM, PEI 200 nM |
| Ad + PEI + PSS + PEI + PSS | PEI 180 nM, PSS 200 nM, PEI 200 nM, PSS 200 nM |
| Ad + PS | PS 320 nM |
| Ad + Poly-L-arginine | Poly-L-arginine 300 nM |
| Ad + Lipofecten | Lipofecten 200 nM |
| Ad + Lipofecten + Alginate | Lipofecten 200 nM, Alginate 25 um |
| Ad + Lipofectamine | Lipofectamine 200 nM |
| Ad + Lipofectamine + Alginate | Lipofectamine 200 nM, Alginate 25 um |
| Ad + PAH | PAH 400 nM |
| Ad + PAH + PSS | PAH 400 nM, PSS 200 nM, |
| Ad + PAH + PSS + PAH | PAH 400 nM, PSS 200 nM, PAH 400 nM |
| Ad + PAH + PSS + PAH + PSS | PAH 400 nM, PSS 200 nM, PAH 400 nM, PSS 200 nM |
| Ad + Chitosan | Chitosan 50 uM, |
| Ad + Chitosan + Hyaluronic acid | Chitosan 50 uM, Hyaluronic acid 1 uM |
| Ad + Chitosan + Hyaluronic acid + Chitosan | Chitosan 50 uM, Hyaluronic acid 1 uM, Chitosan 50 uM |
| Ad + Chitosan + Hyaluronic acid + Chitosan + | Chitosan 50 uM, Hyaluronic acid 1 uM, Chitosan 50 uM, Hyaluronic |
| Hyaluronic acid | acid 1 uM |
| Ad + Chitosan | Chitosan 50 uM, |
| Ad + Chitosan + Alginate | Chitosan 50 uM, Alginate 25 uM |
| Ad + Chitosan + Alginate + Chitosan | Chitosan 50 uM, Alginate 25 uM, Chitosan 50 uM, |
| Ad + Chitosan + Alginate + Chitosan + Alginate | Chitosan 50 uM, Alginate 25 uM, Chitosan 50 uM, Alginate 25 uM |

### Formulations

In some embodiments, the present invention provides a formulation comprising a biological material (e.g., viral particles) having a nanocoating thereon and an entity selected from the group consisting of a cryoprotectant, a surfactant, or a buffer, or combinations thereof. For example, a formulation may comprise viral particles having a nanocoating thereon and a cryoprotectant. A formulation may comprise viral particles having a nanocoating thereon, a cryoprotectant, and a surfactant. A formulation may comprise viral particles having a nanocoating thereon, a cryoprotectant, and a buffer. A formulation may comprise viral particles having a nanocoating thereon, a cryoprotectant, a surfactant, and a buffer. A formulation may comprise viral particles having a nanocoating thereon, a surfactant, and a buffer. In some embodiments, a formulation excludes a cryoprotectant. In some embodiments, a formulation excludes a surfactant. In some embodiments, a formulation excludes a buffer.

A liquid formulation can be an aqueous formulation, in some embodiments. Typically the formulations are prepared with purified water, e.g., deionized water or pharmaceutical grade water-for-injection (WFI). However, in some embodiments an aqueous formulation can comprise water and one or more solvents which are miscible with water (e.g., ethanol, dimethyl sulfoxide, or any other pharmaceutically acceptable solvent or combination of solvents).

In certain embodiments, the present formulations are buffered. Buffering of the present formulations can provide desirable characteristics or properties, e.g., stabilization of the biological materials (e.g., viral particles) in the present formulations before, during and/or after coating and/or drying. Suitable buffers include any pharmaceutically acceptable buffering agents. For example, suitable buffering agents include phosphoric acid/phosphate salts, citric acid/citrate salts, HEPES and salts thereof, and combinations of pharmaceutically acceptable acids or salts, etc., In some embodiments, the buffer comprises sodium citrate, a phosphate salt (e.g., sodium, potassium, etc.), tris(hydroxymethyl)aminomethane (TRIS) and/or 3-(N-morpholino)propanesulfonic acid (MOPS). In one embodiment, the present formulation is an aqueous formulation comprising mono- and dibasic sodium phosphate, and the formulation has a pH of about 4 to about pH 7.5, about pH 4 to about pH 6, or about pH 5. In some embodiments, the pH is about 7.5.

The concentration or identity of the buffering agent(s) employed van be selected to provide a desired level of pH stability of the present formulations. Typical concentrations of buffer in the present formulations can range from about 0.001 M to about 1 M. In some embodiments, the amount is about 0.001 M, about 0.01 M, about 0.1 M, about 0.2 M, about 0.3 M, about 0.4 M, about 0.5 M, about 0.6 M, about 0.7 M, about 0.8 M, about 0.9 M, or any other value or range of values therein. In one embodiment, the concentration of buffering agent is about 0.2 M. Suitable buffering agents or buffer concentrations or formulation pH levels can be selected to provide desired formulation properties. For example, the amount of buffer employed in the present formulations can be selected to provide an optimum pH level for stabilizing the viral particles. In some embodiments, the pH ranges from about pH 3 to about pH 8. In some embodiments, the pH ranges from about pH 4 to about pH 7. In some embodiments, the pH ranges from about pH 4 to about pH 6.

Drying of the present nanocoated biological materials (e.g., viral particles) can be effected by spray-drying, freeze drying, vacuum drying, etc. Typically, the present nanocoated biological materials (e.g., viral particles) have similar activities (e.g., infectivities) after coating or drying. In some embodiments, nanocoated biological materials (e.g., viral particles) after coating or drying have at least 70%, at least 75%, at least 80%, at least 85%, at least 90%, at least 95%, or at least 98% (or any other value or range of values therein or thereabove) of infectivities or activities prior to coating or drying. In some embodiments, nanocoated viral particles have at least 70% of infectivity prior to coating or drying. In some embodiments, the nanocoated viral particles have at least 90% of infectivity prior to coating or drying. In some embodiments, the nanocoated viral particles have at least 95% of infectivity prior to coating or drying.

The present formulations can comprise one or more cryoprotectants. Such cryoprotectants can stabilize the nanocoated biological materials (e.g., viral particles) in the present formulations and in the dried powders formed from the present formulations. As such, the amount of a cryoprotectant used is sufficient to impart stability of the coated biological material (e.g., viral particle) compared to the biological material alone. Any physiologically and pharmaceutically acceptable cryoprotectants can be used. Exemplary cryoprotectants include sucrose, raffinose, maltodextrin, stachyose, lactose, starch, trehalose, glucose, cyclodextrin (e.g., α-cyclodextrin, β-cyclodextrin, γ-cyclodextrin or mixtures thereof), mannitol, dextrose and carboxymethyl cellulose. In some embodiments, the cryoprotectants are β-cyclodextrin and sucrose. In some embodiments, the cryoprotectant is maltodextrin. Suitable cryoprotectants include, but are not limited to, monosaccharides, disaccharides, trisaccharides, oligosaccharides, polysaccharides, and their corresponding sugar alcohols, etc. Both natural and synthetic cryoprotectants are suitable for use in the present formulations. In some embodiments, the cryoprotectant is not a saccharide. In some embodiments, the cryoprotectant is not a monosaccharide. In some embodiments, the cryoprotectant is not glucose.

Synthetic cryoprotectants suitable for use in the present formulations include, but are not limited to, carbohydrates which have the glycosidic bond replaced by a thiol or carbon bond. Both D and L forms of a carbohydrate can be used. The carbohydrate can be non-reducing or reducing. Exemplary reducing carbohydrates suitable for use in the present formulations include, but are not limited to, glucose, maltose, lactose, fructose, galactose, mannose, maltulose, iso-maltulose and lactulose. Exemplary non-reducing carbohydrates include, but are not limited to, trehalose, raffinose, stachyose, sucrose and dextran. Other useful carbohydrates can include non-reducing glycosides of polyhydroxy compounds selected from sugar alcohols and other straight chain polyalcohols. The sugar alcohol glycosides can be monoglycosides, e.g., compounds obtained by reduction of disaccharides such as lactose, maltose, lactulose and maltulose. Further, any of the above-described carbohydrate(s) can be hydrates.

The amount of cryoprotectant in the present formulations can range from about 0.1 wt % to about 50 wt %. In some embodiments, the amount is about, at least about, or at most about 1 wt %, 2 wt %, 3 wt %, 4 wt %, 5 wt %, about 6 wt %, 7 wt %, 8 wt %, 9 wt %, 10 wt %, 15 wt %, 20 wt %, 25 wt %, 30 wt %, 35 wt %, 40 wt %, 45 wt %, or any other value or range of values therein. In some embodiments, the amount is less than 5 wt %. In some embodiments, the amount is less than 4%. In some embodiments, the amount is greater than 5 wt %. In some embodiments, the amount is greater than 10 wt %. In some embodiments, the amount ranges from about 1 wt % to about 10 wt %, about 6 wt % to about 20 wt %, about 7 wt % to about 50 wt %, about 10 wt % to about 50 wt %, about 20 wt % to about 50 wt %, or about 10 wt % to about 40 wt %. In some embodiments, use of a cryoprotectant is necessarily accompanied by use of a surfactant and/or a buffer. In certain embodiments, the cryprotectants include β-cyclodextrin and sucrose, and the amount of β-cyclodextrin and sucrose in the formulation is about 5 wt %, about 6 wt %, about 7 wt %, about 8 wt %, about 9 wt %, about 10 wt %, about 11 wt %, about 12 wt %, about 13 wt %, about 14 wt %, or about 15 wt %. In certain embodiments, the cryoprotectants include glucose and lactose, and the amount of glucose and lactose in the formulation is about 10 wt %. In certain other embodiments, the cryoprotectants include raffinose and sucrose, and the amount of raffinose and sucrose in the formulation is about 11 wt %.

To improve the antigenicity of the coated viral particles, one or more adjuvants can be included in the mixture that is spray dried to create the present viral particle-containing powders. Examples of such adjuvants include, but are not limited to, salts, such as calcium phosphate, aluminum phosphate, calcium hydroxide, aluminum hydroxide and magnesium chloride; natural polymers such as algal glucans (e.g., beta glucans), chitosan or crystallized inulin, gelatin; synthetic polymers such as polylactides, polyglycolides, polylacitide coglycolides or methylacrylate polymers; micelle-forming cationic or non-ionic block copolymers or surfactants such as Pluronics, L121, 122 or 123, Tween 80, or NP-40; fatty acid, lipid or lipid and protein based vesicles such as liposomes, proteoliposomes, ISCOM and cochleate structures; stabilizers such as triethyl citrate, and surfactant stabilized emulsions composed of synthetic or natural oils and aqueous solutions. Persons of skill in the art will be familiar with appropriate amounts of adjuvant to employ. In some embodiments, the concentration of a salt as an adjuvant ranges from about 0.1 mM to about 100 mM.

Further, or to improve the properties of the present formulations or powders produced therefrom, one or more pharmaceutical excipients or other additives can be present in the present formulations. Such excipients or additives can include one or more stabilizing polyols, e.g., higher polysaccharides/polymers (for promoting controlled release), magnesium stearate, leucine or trileucine (as lubricants), and phospholipids or surfactants. Blowing agents, e.g., volatile salts such as ammonium carbonate, formic acid, etc. can also be included in the feedstock to produce reduced density particles in the present spray dried powders.

Spray aids can also be employed in the present formulation. Such spray aids can reduce the viscosity and/or improve the fluid mechanical characteristics of the present formulations during a spray drying process. Spray aids include maltodextrin, lactose, gelatin, talc, triethylcitrate, and mixtures thereof. Such spray aids can be present in the formulations in amounts ranging from about 1 wt % to about 15 wt %. In some embodiments, the amount is about, at most about, or at least about 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, or any other value or range of values therein. In certain embodiments, the spray aid is maltodextrin, and the amount of maltodextrin in the formulation is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %. In other embodiments, the spray aid is lactose, and the amount of lactose in the formulation is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %. In still other embodiments, the spray aid is gelatin, and the amount of gelatin in the formulation is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %.

Surfactants can also be employed in the present formulations. For example, surfactants include polysorbates, poloxamers and mixtures thereof. In certain embodiments, the surfactant is selected from pluronic F-68, Tween 80, BRIJ 35, and mixtures thereof. The surfactant(s) can comprise from about 0.01 wt % to about 2 wt % of the present formulations. In some embodiments, the amount is about, at most about, or at least about 0.02 wt %, 0.04 wt %, 0.06 wt %, 0.08 wt %, 0.1 wt %, 0.2 wt %, 0.3 wt %, 0.4 wt %, 0.5 wt %, 1.0 wt %, 1.5 wt %, or any other value or range of values therein. In some embodiments, the range is about 0.01% to about 1 wt %, about 0.05%, to about 1.5 wt %, about 0.05% to about 1 wt %, or about 1 wt % to about 2 wt %.

In some embodiments, the present formulations can comprise one or more enteric polymers. Drying (e.g., freeze or spray drying) of formulations comprising an enteric polymer allows simultaneous drying and coating of the present nanocoated viral particles. Accordingly, the present formulations can comprise from about 0.1 wt % to about 5 wt % of an enteric polymer. In some embodiments, the amount is about, at most about, or at least about 0.2 wt %, 0.4 wt %, 0.6 wt %, 0.8 wt %, 1 wt %, 2 wt %, 3 wt %, 4 wt %, or any other value or range of values therein. Representative examples of enteric polymers which can be useful in the present formulations include esters of cellulose and its derivatives (e.g., cellulose acetate phthalate, hydroxypropyl methylcellulose phthalate, hydroxypropyl methylcellulose acetate succinate), polyvinyl acetate phthalate, pH-sensitive methacrylic acid-methacrylate copolymers and shellac. In one embodiment, the enteric polymer is Eudragit® L 30 D-55. In certain embodiments, the enteric polymer is Eudragit® L 30 D-55, and is present in the formulation at about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %. In some embodiments, the enteric polymer is hydroxypropyl methylcellulose phthalate, and is present in the formulation at about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %. In other embodiments, the enteric polymer is cellulose acetate phthalate, and is present in the formulation at about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %. Enteric polymers are widely used in the pharmaceutical industry for encapsulation of an active drug, for taste masking, and/or for immediate and/or modified release formulations, either alone or in combination with one or more other polymers (e.g., water insoluble polymers, water soluble polymers, etc.). Modified-release formulations can include, e.g., delayed-release, extended-release, site-specific targeting and receptor targeting. Such modified release formulations are known and will be appreciated by those skilled in the art.

Eudragit® or other comparable enteric polymers as described herein are available as aqueous or organic solutions or as powders. The use of enteric or other suitable polymers can facilitate the formation of dried powders wherein nanocoated viral particles and any additives or excipients can be encapsulated or coated with an enteric polymer to yield a free-flowing and non-dusty powder suitable for direct tablet compression. Accordingly, enteric coating of nanocoated virus particles by drying (e.g., spray-drying) of formulations comprising an enteric polymer can have several advantages in an exemplary dosage manufacturing process.

One exemplary formulation comprises cryoprotectants sucrose and fructose, spray aid gelatin, glycerin, surfactant poloxamer, phosphate buffer and magnesium chloride. Glycerin (glycerol) may be comprised in any formulation described herein. Another exemplary formulation comprises cryoprotectants sucrose and β-cyclodextrin, spray aid maltodextrin, glycerin, a surfactant mixture of polysorbate and poloxamer, phosphate buffer and magnesium chloride (e.g., 10 mM MgCl₂). Still another exemplary formulation comprises galactose and β-cyclodextrin, spray aid talc, glycerin, polysorbate, phosphate buffer and magnesium chloride. Another exemplary formulation comprises sucrose, magnesium chloride, phosphate buffer, and glycerin.

One exemplary formulation comprises about 10 wt % sucrose and β-cyclodextrin, about 7 wt % maltodextrin, about 0.4 wt % glycerin, from about 0.2 to about 0.5 wt % polysorbate, a phosphate buffer concentration of about 200 mM and a magnesium chloride concentration of about 10 mM. Another exemplary formulation comprises about 12 wt % sucrose and β-cyclodextrin, about 5 wt % maltodextrin, about 4 wt % glycerin, from about 0.2 to about 0.5 wt % polysorbate, a phosphate buffer concentration of about 200 mM and a magnesium chloride concentration of about 10 mM. Still another exemplary formulation comprises about 14 wt % sucrose and β-cyclodextrin, about 3 wt % maltodextrin, about 0.4 wt % glycerin, from about 0.2 to about 0.5 wt % polysorbate, a phosphate buffer concentration of about 200 mM and a magnesium chloride concentration of about 10 mM.

One exemplary formulation comprises about 2 wt % sucrose and β-cyclodextrin, about 14 wt % maltodextrin, about 0.4 wt % glycerin, from about 0.2 to about 0.5 wt % polysorbate, about 0.4 wt % Eudragit® L 30 D-55, a phosphate buffer concentration of about 200 mM and a magnesium chloride concentration of about 10 mM. Another exemplary formulation comprises about 4 wt % sucrose and β-cyclodextrin, about 12 wt % maltodextrin, about 0.4 wt % glycerin, from about 0.2 to about 0.5 wt % polysorbate, about 0.5 wt % Eudragit® L 30 D-55, a phosphate buffer concentration of about 200 mM and a magnesium chloride concentration of about 10 mM. Another exemplary formulation comprises about 6 wt % sucrose and β-cyclodextrin, about 10 wt % maltodextrin, about 0.4 wt % glycerin, from about 0.2 to about 0.5 wt % polysorbate, about 0.6 wt % Eudragit® L 30 D-55, a phosphate buffer concentration of about 200 mM and a magnesium chloride concentration of about 10 mM.

Table 2 presents additional non-limiting examples of combinations of cationic material, anionic material, cryoprotectant, surfactant, and/or buffer, where additional components can be added to any of these exemplary combinations (e.g., excipients, additives, enteric polymers, etc.). Any of column A can be combined with any of columns B, C, D, and/or E. For example, any of A can be combined with any of B, any of D, and any of E. In some embodiments, A1, B1, C1, D1, and E1 are combined. Other exemplary embodiments include: A1+B2+C5+D3+E1; A1+B5+B10+D1+E6; A8+B5+D1+E2; A9+B6+C3+D1+E7; A10+B8+C2+D2; A11+B1+C4; A12+B7+E6; A11+B11+D1; A12+B7+E6; A13+C5+D1+E5; A14+B2+E10; A18+B3+C4+E2; A22+B5+B9+C1; A22+C1.

| A - Nanocoating | | B - Cryoprotectant | | C - Surfactant | | D - Buffer | | E - Adjuvant | |
|---|---|---|---|---|---|---|---|---|---|
| A1 | PEI | B1 | monosaccharide | C1 | polysorbate | D1 | phosphate | E1 | salt |
| A2 | PEI + BSA | B2 | disaccharide | C2 | poloxamer | D2 | citrate | E2 | natural polymer |
| A3 | PEI + BSA + PEI | B3 | trisaccharide | C3 | Pluronic F-68 | D3 | HEPES | E3 | synthetic polymer |
| A4 | PEI + BSA + PEI + BSA | B4 | monoglycoside | C4 | Tween 80 | | | E4 | calcium phosphate |
| A5 | PEI + PSS | B5 | sucrose | C5 | polysorbate + | | | E5 | aluminum hydroxide |
| | | | | | poloxamer | | | | |
| A6 | PEI + PSS + PEI | B6 | trehalose | | | | | E6 | magnesium chloride |
| A7 | PEI + PSS + PEI + PSS | B7 | raffinose | | | | | E7 | chitosan |
| A8 | PS | B8 | stachyose | | | | | E8 | gelatin |
| A9 | Poly-L-arginine | B9 | dextran | | | | | E9 | polylactide |
| A10 | Lipofecten | B10 | β-cyclodextrin | | | | | E10 | liposome |
| A11 | Lipofecten + Alginate | B11 | maltodextrin | | | | | | |
| A12 | Lipofectamine | | | | | | | | |
| A13 | Lipofectamine + Alginate | | | | | | | | |
| A14 | PAH | | | | | | | | |
| A15 | PAH + PSS | | | | | | | | |
| A16 | PAH + PSS + PAH | | | | | | | | |
| A17 | PAH + PSS + PAH + PSS | | | | | | | | |
| A18 | Chitosan | | | | | | | | |
| A19 | Chitosan + Hyaluronic acid | | | | | | | | |
| A20 | Chitosan + Hyaluronic acid + | | | | | | | | |
| | Chitosan | | | | | | | | |
| A21 | Chitosan + Hyaluronic acid + | | | | | | | | |
| | Chitosan + Hyaluronic acid | | | | | | | | |
| A22 | Chitosan + Alginate | | | | | | | | |
| A23 | Chitosan + Alginate + | | | | | | | | |
| | Chitosan | | | | | | | | |
| A24 | Chitosan + Alginate + | | | | | | | | |
| | Chitosan + Alginate | | | | | | | | |

The total solids content of the present formulations can range from about 5 wt % to about 30 wt % (e.g., about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, or any other value or range of values therein). In some embodiments, the total solids content is less than about 30 wt %. In other embodiments, the total solids content is less than about 10 wt %. In some embodiments, the amount ranges between about 5 wt % and about 25 wt %.

The present formulations are generally prepared by sequentially adding and dissolving each component in solution (e.g., to WFI or DI water), mixing the resultant solution until a given component is dissolved, then adding the next component. However, in certain embodiments, it can be advantageous to pre-mix certain components separately in two or more solutions, and then combine the two or more solutions to obtain a final formulation for either direct use, storage, or subsequent drying. Alternatively, it can be advantageous to add the various components of the mixture in a certain order such that the resultant solution is substantially homogenous.

Nanocoated biological materials (e.g., viral particles) produced from drying the present formulations will generally have a composition which reflects the proportions of non-volatile components in the spray drying solution from which they were formed. Dried particles produced from the present formulations will have a composition which corresponds to that of the formulation form which they are spray dried, less any volatile components removed during the spray drying process (e.g., water). Particles produced by spray drying the present formulations are typically substantially homogeneous, both within a particle and from particle to particle. Particle morphology is generally spherical. In some embodiments, the present particles can be substantially solid. In other embodiments, the particles can be hollow spheres. Morphology of the present particles can be controlled by adjusting various drying parameters or formulation parameters, as known to those skilled in the art.

Particles produced from the present formulation can have a size ranging from about 0.01 μm to about 500 μm. In some embodiments, the range is from about 0.01 to about 400 μm, from about 0.1 μm to about 300 μm, from about 0.5 μm to about 200 μm, from about 1 μm to about 100 μm, from about 1 μm to about 75 μm, from about 2 μm to about 50 μm, from about 2 μm to about 20 μm, from about 2 μm to about 10 μm, or any other value or range of values therein. When powders produced by according to the present invention are intended for pulmonary delivery, particles having a mean diameter ranging from about 0.1 μm to about 10 μm can be employed for deep lung deposition. Alternatively, if the powder is intended for nasal delivery, particle diameter can be larger, e.g., from about 10 μm to about 75 μm. Particle size in the present powders can be measured, for example, using an Horiba LA-950 laser diffraction dry powder feeder apparatus or an equivalent means of measuring.

Furthermore, particle sizes can be reported as a statistical distribution. For example, where particle diameter is reported as about 15 μm, that measurement can reflect some fraction of the total distribution having a particle size of about 15 μm. Thus, the a distribution or “d” value can report some percentage of the whole having the reported size. For example, a d80 value of 15 μm particle diameter reports that approximately 80% of the particles measured have a diameter of about 15 μm. Similarly, d70, d75, d85, d90, d95 (or any other value or range of values therein or thereabove) values, can be used. Such determination of particle size and statistical distributions associated therewith are known and will be appreciated by those skilled in the art.

When the present formulations are spray-dried, they can advantageously be spray-dried at lower temperatures relative to conventional compositions for spray-drying viral particles. Thus, in some embodiments, the present formulations are spray dried with an inlet temperature of less than about 70° C., less than about 60° C., less than about 50° C., less than about 40° C., less than about 35° C., less than about 30° C., or any other value or range of values therein or therebelow. Accordingly, in certain embodiments, the present formulations are spray dried with an outlet temperature of less than about 50° C., less than about 40° C., less than about 30° C., less than about 25° C., less than about 20° C., or any other value or range of values therein or therebelow. Such reduced inlet and outlet temperatures advantageously enable spray-drying of the present nanocoated viral particles without significant loss of viral infectivity upon drying.

The present formulations are also suitable for large-scale production of spray dried viral powders with, e.g., commercial spray-drying equipment. Thus, in certain embodiments, the present formulations can be spray dried at production rates of at least about 50 g/hr, about 60 g/hr, about 70 g/hr, about 80 g/hr, about 90 g/hr, about 100 g/hr, about 120 g/hr, about 140 g/hr, about 160 g/hr, about 180 g/hr, about 200 g/hr, about 220 g/hr, about 240 g/hr, about 260 g/hr, about 280 g/hr, about 300 g/hr, about 350 g/hr, about 400 g/hr, about 450 g/hr, about 500 g/hr, or any other value or range of values therein or thereabove. For example, the production rate can be about 50 g/hr to about 500 g/hr. Accordingly, in certain embodiments, the present methods provide large-scale drying of the present formulations at reduced temperatures (e.g., less than about 40° C.).

Particles produced from the present formulations can be dried (e.g., spray or freeze dried) such that they have a specific moisture content after spray drying. For example, the present spray dried powders can have a moisture content of less than about 10 wt % (e.g., less than about 9 wt %, less than about 8 wt %, less than about 7 wt %, less than about 6 wt %, less than about 5 wt %, less than about 4 wt %, less than about 3 wt %, less than about 2 wt %, less than about 1 wt %, or any other value or range of values therein or therebelow). In some embodiments, the moisture content ranges from about 1% to about 10%. Moisture levels can be determined as known by skilled artisans, such as by using Karl Fisher titration.

It will be appreciated by the skilled person that the present formulations or dried particles are to be formulated to contain physiologically effective amounts of biological materials (e.g., viral particles). That is, when the formulations or dried powders are delivered in a unit dosage form, there should be a sufficient amount of biological material (e.g., viral particle) to achieve a desired therapeutic response.

In some embodiments, the biological materials in the formulations described herein can be more stable than naked biological materials with respect to one or more stability properties (chemical, thermal, mechanical, etc.). For example, a liquid (e.g., aqueous) formulation comprising viral particles can be dried to provide a dried powder having viral particles that are more stable (e.g., chemically, thermally, mechanically, etc.) than the naked viral particles. In some embodiments, biological materials (e.g., viral particles) in a formulation can be more stable than in a formulation lacking a cryoprotectant, a surfactant and/or a buffer, such as with respect to chemical stability, thermal stability, mechanical stability, etc.

In some embodiments of the present invention, formulations comprising the present nanocoated biological materials (e.g., viral particles) or dried powders produced from the present formulations can be used directly. In some embodiments, the present dried powders or formulations can be used as a component in the preparation of vaccines. For example, the present invention provides vaccines comprising one or more viral vectors in the present formulations or dried powders. In certain embodiments, the viral vector is an adenoviral vector. In some other embodiments, the viral vector is a virus. In yet other embodiments, the viral vector includes the viral genome alone and does not include a viral capsid.

In certain embodiments, a vaccine of the invention, upon administration to a subject, is capable of stimulating an immune response (e.g., an innate immune response, humoral immune response, cellular immune response, or all three) in the subject. In certain embodiments, the immune response includes a measurable response (e.g., a measurable innate, humoral or cellular immune response, or combination thereof) to an epitope encoded by a heterologous sequence inserted or integrated into an adenoviral vector of the vaccine. In certain embodiments, a vaccine of the invention is capable of providing protection against an infectious pathogen or against cancer. For example, in certain embodiments, the vaccine is capable of stimulating an immune response against one or more antigens (e.g., encoded by a heterologous sequence) such that, upon later encountering such an antigen, the subject receiving the vaccine has an immune response that is stronger than it would have been if the vaccine had not been administered previously.

In some embodiments, a vaccine of the invention is capable of providing protection against an infectious pathogen or cancer in a subject with pre-existing immunity to adenovirus. In other embodiments, a vaccine of the invention is capable of ameliorating a pathogen infection or cancer or reducing at least one symptom of a pathogen infection or cancer. For instance, in one embodiment, the vaccine of the invention induces a therapeutic immune response against one or more antigens encoded by a heterologous sequence such that symptoms or complications of a pathogen infection or cancer will be alleviated, reduced, or improved in a subject suffering from such an infection or cancer.

The present vaccines can be prepared and formulated as a pharmaceutical composition for administration to a mammal in accordance with techniques well known in the art. Formulations for oral administration can consist of capsules or tablets containing a predetermined amount of the present spray-dried powders; liquid solutions comprising the present spray-dried powder dissolved in an ingestible diluent such as water, saline, orange juice, and the like; suspensions in an appropriate liquid; and suitable emulsions.

The present vaccines can, for example, be formulated as enteric coated capsules or tablets for oral administration in order to bypass the upper respiratory tract and allow viral replication in the gut. See, e.g., Tacket et al., Vaccine 10:673-676, 1992; Horwitz, in Fields et al., eds., Fields Virology, third edition, vol. 2, pp. 2149-2171, 1996; Takafuji et al., J. Infec. Dis. 140:48-53, 1979; and Top et al., J. Infec. Dis. 124:155-160, 1971. Alternatively, in the case of powders comprising enteric coated nanocoated viral particles as previously described herein, such enteric powders can be directly compressed to provide oral, enteric coated formulations. Alternatively, the vaccine can be formulated as a pharmaceutical composition in conventional solutions, such as sterile saline, and can incorporate one or more pharmaceutically acceptable carriers or excipients. The pharmaceutical composition can further comprise other active agents.

In certain embodiments, formulations of the invention comprise a buffered solution comprising the present dried powders in a pharmaceutically acceptable carrier. A variety of carriers can be used, such as buffered saline, water and the like. Such solutions are generally sterile and free of undesirable matter. These compositions can be sterilized by conventional, well-known sterilization techniques, or can be sterile filtered. The compositions can contain pharmaceutically acceptable auxiliary substances as required to approximate physiological conditions such as pH adjusting and buffering agents, tonicity adjusting agents and the like, for example, sodium acetate, sodium chloride, potassium chloride, calcium chloride, sodium lactate and the like. Alternatively, the present formulations can also be combined with diluents or excipients as described herein to produce a dosage form.

Pharmaceutically acceptable carriers can contain a physiologically acceptable compound that acts, e.g., to stabilize the composition or to increase or decrease the absorption of the virus or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipient, or other stabilizers or buffers. Detergents can also be used to stabilize the composition or to increase or decrease absorption. One skilled in the art will appreciate that the choice of a pharmaceutically acceptable carrier, including a physiologically acceptable compound depends, e.g., on the route of administration of the present powders and on the particular physio-chemical characteristics of any co-administered agent.

The present nanocoated biological materials (e.g., viral particles) can also be administered in a lipid formulation, more particularly either complexed with liposomes or to lipid/nucleic acid complexes or encapsulated in liposomes. The vectors of the current invention, alone or in combination with other suitable components, can also be made into aerosol formulations to be administered via inhalation. The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, e.g., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. In some embodiments, vaccines of the invention can be formulated as suppositories, for example, for rectal or vaginal administration.

Vaccines can have a unit dosage comprising between about 10³ to about 10¹³ (e.g., about 10³ to about 10⁴, about 10⁴ to about 10⁵, about 10⁵ to about 10⁶, about 10⁶ to about 10⁷, about 10⁷ to about 10⁸, about 10⁸ to about 10⁹, about 10⁹ to about 10¹⁰, about 10¹⁰ to about 10¹¹, about 10¹¹ to about 10¹², about 10¹² to about 10¹³) recombinant adenoviruses in a single dose. The dosages can vary based on the route of administration. For instance, vaccines formulated for sublingual or intranasal administration can contain a lower dosage of adenovirus per single dose than vaccines formulated for oral administration. One of skill in the art can determine the appropriate dosage for a particular patient depending on the type of infection or cancer, and the route of administration to be used without undue experimentation.

The vaccines of the invention can be administered alone, or can be co-administered or sequentially administered with other immunological, antigenic, vaccine, or therapeutic compositions. Such compositions can include other agents to potentiate or broaden the immune response, e.g., IL-2 or other cytokines which can be administered at specified intervals of time, or continuously administered (see, e.g., Smith et al., N Engl J Med 1997 Apr. 24; 336(17):1260-1; and Smith, Cancer J Sci Am. 1997 December; 3 Suppl 1:S137-40). The vaccines or vectors can also be administered in conjunction with other vaccines or vectors. For example, a vaccine of the invention can be administered either before or after administration of an adenovirus of a different serotype. A vaccine preparation can also be used, for example, for priming in a vaccine regimen using an additional vaccine agent.

The present vaccines can be delivered systemically, regionally, or locally. Regional administration refers to administration into a specific anatomical space, such as intraperitoneal, intrathecal, subdural, or to a specific organ, and the like. Local administration refers to administration of a composition into a limited, or circumscribed, anatomic space such as an intratumor injection into a tumor mass, subcutaneous injections, intramuscular injections, and the like. Those skilled in the art will appreciate that local administration or regional administration can also result in entry of the viral preparation into the circulatory system. Delivery routes include parenteral administration, e.g., intradermal, intramuscular or subcutaneous routes. Other routes include oral administration, including administration to the oral mucosa (e.g., tonsils), intranasal, sublingual, intravesical (e.g., within the bladder), rectal, and intravaginal routes. For delivery of adenovirus, administration can often be performed via inhalation. Aerosol formulations can, for example, be placed into pressurized, pharmaceutically acceptable propellants, such as dichlorodifluoro-methane, nitrogen and the like. They can also be formulated as pharmaceuticals for non-pressurized preparations such as in a nebulizer or an atomizer. Typically, such administration is in an aqueous pharmacologically acceptable buffer as described above. Delivery to the lung can also be accomplished, for example, using a bronchoscope.

The vaccines of the invention can be administered in a variety of unit dosage forms, depending upon the intended use, e.g., prophylactic vaccine or therapeutic regimen, and the route of administration. With regard to therapeutic use, the particular condition or disease and the general medical condition of each patient will influence the dosing regimen. The concentration of adenovirus in the pharmaceutically acceptable excipient can be, e.g., from about 10³ to about 10¹³ virus particles per dose, between about 10⁴ to about 10¹¹ virus particles per dose, between about 10⁶ to about 10¹⁰ virus particles per dose, between about 10⁷ to about 10⁹ virus particles per dose, or between about 10⁹ to about 10¹¹ virus particles per dose. In other embodiments, the concentration of adenovirus in the pharmaceutically acceptable excipient can be, e.g., from about 10³ to about 10⁹, about 10⁴ to about 10⁸, or about 10⁵ to about 10⁷ infectious units per dose.

The replication-competent adenoviral vectors of the invention are typically administered at much lower doses than would be needed to achieve equivalent expression levels of the encoded transgene by a replication-defective adenovirus recombinant administered in vivo. Replication competent adenovirus vectors can be administered at a range of dosages (see, e.g., U.S. Pat. No. 4,920,209; Smith et al., J. Infec. Dis. 122:239-248, 1970; Top et al., J. Infect. Dis. 124:155-160, 1971; Takafuji et al., J. Infec. Dis. 140:48-53, 1979; Tacket et al., Vaccine 10:673-676, 1992). For example, 10⁴ to 10⁹ 50% tissue culture infective doses (or plaque forming units) can be administered. Typically an oral dosage for a replication-competent adenovirus is about 10^{4.6} 50% tissue culture infective doses or 10⁷ particles. In some embodiments, an oral dosage for a replication-competent adenovirus is about 10¹¹ particles. Typical intranasal administration of adenovirus recombinants is often in dosages of about 10³ to about 10⁵ plaque forming units. The exact concentration of virus, the amount of formulation, and the frequency of administration can also be adjusted depending on the levels of in vivo, e.g., in situ transgene expression and vector retention after an initial administration.

The amount and concentration of a biological material (e.g., a viral particle) and the formulation of a given dose, or a “therapeutically effective” dose can be determined by the clinician. A therapeutically effective dose of an adenoviral vaccine, for example, is an amount of adenovirus that will stimulate an immune response to the protein(s) encoded by the heterologous nucleic acid included in the viral vector. The dosage schedule, i.e., the dosing regimen, will depend upon a variety of factors, e.g., the general state of the patient's health, physical status, age and the like. The state of the art allows the clinician to determine the dosage regimen for each individual patient. Adenoviruses have been safely used for many years for human vaccines. See, e.g., Franklin et al., supra; Jag-Ahmade et al., J. Virol., 57:267, 1986; Ballay et al., EMBO J. 4:3861, 1985; PCT publication WO 94/17832. These illustrative examples can also be used as guidance to determine the dosage regimen when practicing the methods of the invention.

Single or multiple administrations of adenoviral formulations can be administered as prophylactic or therapeutic vaccines. In one embodiment, multiple doses (e.g., two or more, three or more, four or more, or five or more doses) are administered to a subject to induce or boost a protective or therapeutic immune response. The two or more doses can be separated by periodic intervals, for instance, one week, two week, three week, one month, two month, three month, or six month intervals.

Throughout the present specification, the terms “about” or “approximately” can be used in conjunction with numerical values or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

Throughout the present specification, the words “a” or “an” are understood to mean “one or more” unless explicitly stated otherwise. Further, the words “a” or “an” and the phrase “one or more” may be used interchangeably.

In any embodiment herein, the term “comprising” may be substituted with “consisting essentially of” or “consisting of.” For those embodiments reciting “consisting essentially of,” it is noted that components, steps, etc. may be included that do not materially affect the basic and novel aspects of the subject matter at issue. For example, a formulation consisting essentially of viral particles having a nanocoating thereon and one or more entities selected from a cryoprotectant, a surfactant, a buffer, or a combination thereof may include aspects that do not destroy the infectivity or activity of the viral particles. For those embodiments reciting “consisting of,” no additional components, steps, etc. are contemplated. Any component described herein (e.g., an cryoprotectant, a surfactant, a buffer, a spray aid, an adjuvant, an enteric polymer, etc.) may be utilized in any embodiment herein that comprises, consists essentially of, or consists of a formulation.

All documents cited or referred to herein are incorporated herein by reference for all purposes.

Headings used herein are for purposes of clarity and organization and are not intended as limiting.

The following non-limiting examples will illustrate various aspects of the present invention. The examples should, of course, be understood to be merely illustrative of only certain embodiments of the invention and not to constitute limitations upon the scope of the invention which is defined by the claims that are appended at the end of this description.

### Examples

### Nanocoating

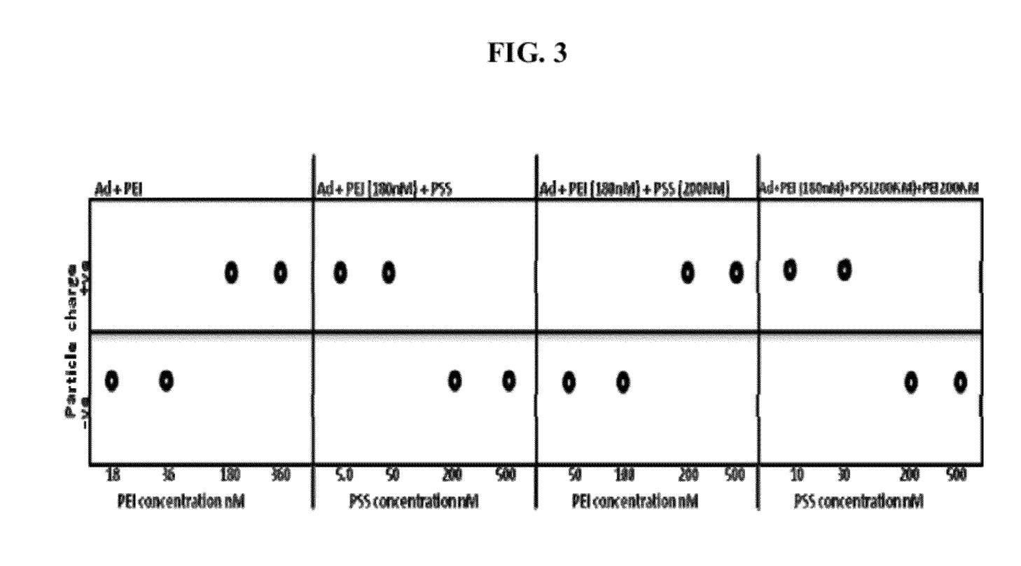

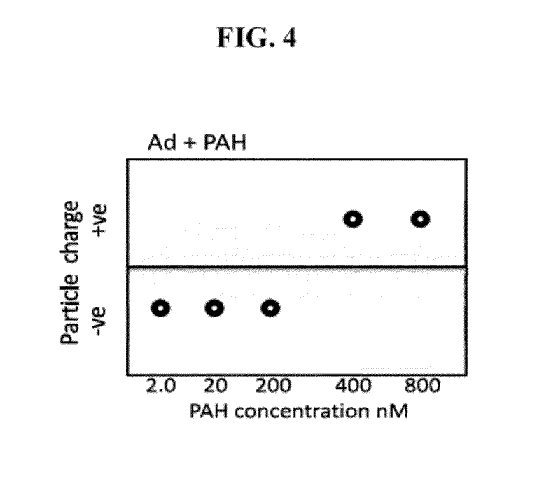

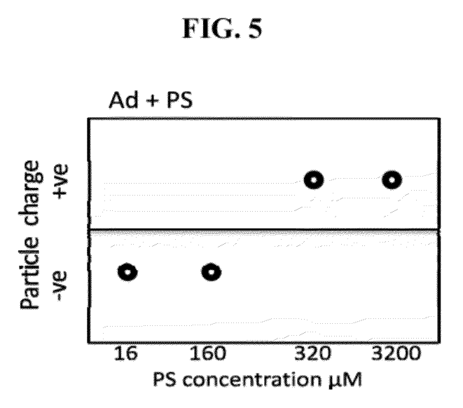

An adenoviral particle suspension is provided in a frozen liquid form containing sucrose (2% wt/v), glycerol (2% w/v), MgCl₂.H₂O (10 mM) and phosphate buffer (KH₂PO₄, 196 mM; K₂HPO₄, 4 mM) at pH 5. After thawing the suspension a known amount of polycation was added to adenovirus particle at a fixed concentration of viral particle per ml (vp/ml) and the mixture was incubated on a rotator for 10 min. After the time of incubation the sample was analyzed for deposition of polycation on the adenovirus particle using anion exchange column chromatography. As the surface charge of the adenovirus particle changes from net negative to net positive with polycation deposition, there is no binding between the column and particle, so the first sample that indicates no binding to the column at a set concentration of polycation was selected for further polyanion deposition. A similar polycation incubation and analytical approach was followed for polyanions deposition. Such steps were repeated for desired number of bilayers. The results are shown in FIG. 3, FIG. 4, and FIG. 5 for [Ad-(polycation/polyanion)n].

Matrix: [Ad-(PEI/PSS)2]: FIG. 3 shows change in adenovirus particle charge when layers of polyethyleneimine (PEI)-polycation and poly(sodium 4-styrenesulfonate)(PSS)-polyanion are alternated for coating of adenovirus.

Matrix: [Ad-(PAH)]: FIG. 4 shows change in adenovirus particle charge when a layer of poly(allylamine hydrochloride)(PAH)-polycation is coated on adenovirus.

Matrix: [Ad-(PS)]: FIG. 5 shows change in adenovirus particle charge when layer of protamine sulfate (PS)-polycation is coated on adenovirus.

In vitro Infectivity

The in vitro infectivity of A549 cells were measured as an indicator of functionality and activity of several of the present nanoencapsulated adenovirus formulations. The TCID50 assay based on the viral infection of the cells was performed for a combination of nanoencapsulation matrices shown in Table 3 below. It was found that the present anionic and/or cationic ionic material used for nanoencapsulation does not interfere with the infectivity of adenovirus.

TABLE 3
Viral Particle Infectivity of Nanoencapsulated Formulations

| Nanoencapsulated Matrix | Activity by NAS TCID₅₀ |
|---|---|
| [Ad]- Control | Infective |
| [Ad-(PAH)] | Infective |
| [Ad-(PS)] | Infective |
| [Ad-(PEI)] | Infective |
| [Ad-(PEI/PSS)] | Infective |
| [Ad-(PEI/PSS/PEI)] | Infective |
| [Ad-(PEI/PSS/PEI/PSS)] | Infective |

### Zeta Potential

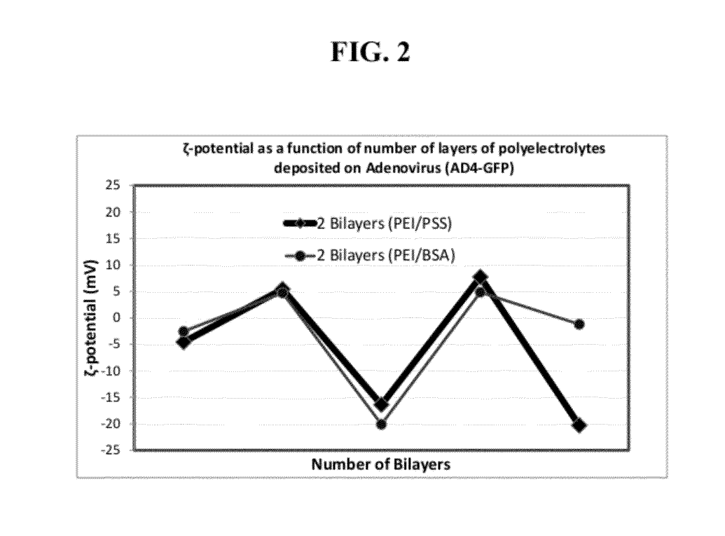

Detection of nanocoating formation on an adenovirus particle was measured using zeta potential with a zeta potential instrument (Malvern, Zetasizer Nano-ZS). The alternating points in FIG. 2 indicate the formation of cationically and anionically charged particles following each coating event.

Those skilled in the art will recognize, or be able to ascertain, using no more than routine experimentation, numerous equivalents to the specific embodiments described specifically in this disclosure. Such equivalents are intended to be encompassed in the scope of the following claims.

What is claimed:

1. A formulation comprising viral particles having a nanocoating thereon and one or more entities selected from a cryoprotectant, a surfactant, a buffer, or a combination thereof.

2. The formulation of claim 1, wherein said viral particles are adenoviral particles.

3. The formulation of claim 1, wherein said viral particles comprise an adenoviral vector that is replication competent.

4. The formulation of claim 1, wherein said nanocoating comprises one or more layers of an anionic or cationic material.

5. The formulation of claim 4, wherein said anionic material comprises poly(sodium 4-styrenesulfonate), hyaluronic acid sodium salt, dextran sulfate sodium salt, bovine serum albumin, polyacrylic acid, polyaspartic acid, polyvinylsulfonate, gelatin, polyacrylamidomethyl propane sulfonic acid, polylactic acid, poly(ethyleneglycol-co-methacrylic acid), alginate, chondroitin sulfate, dioleoylphosphatidylethanolamine, phosphatidylglycerol, phosphatidic acid, phosphatidylserine, or combinations thereof.

6. The formulation of claim 4, wherein said cationic material comprises poly(allylamine hydrochloride), poly(diallyldimethylammonium chloride), poly(ethylene glycol-co-dimethylaminoethyl methacrylate), protamine sulfate, hexadimethrine bromide, chitosan, poly-L-lysine hydrobromide, poly-L-arginine, polyethyleneimine, DEAE-dextran, lipofectin, lipofectamine, dioctadecylamidoglycylspermine, [N—(N9,N9-dimethylaminoethane)carbamoyl]cholesterol, 1,2-di-(9Z-octadecenoyl)-3-trimethylammonium-propane methyl sulfate salt, 1,2-ditetradecanoyl-3-dimethylammonium-propane, dimethyldioctadecylammonium bromide salt, or combinations thereof.

7. The formulation of claim 1, wherein said cryoprotectants comprise sucrose, raffinose, maltodextrin, stachyose, lactose, starch, trehalose, glucose, cyclodextrin, mannitol, dextrose, carboxymethyl cellulose, or combinations thereof.

8. The formulation of claim 7, wherein the cyclodextrin is α-cyclodextrin, β-cyclodextrin, γ-cyclodextrin or a mixture thereof.

9. The formulation of claim 1, wherein said cryoprotectants comprise from about 0.1 wt % to about 50 wt % of said formulation.

10. The formulation of claim 1, wherein said surfactants comprise Pluronic F-68, Tween 80, BRIJ 35, or combinations thereof.

11. The formulation of claim 1, wherein said buffers comprise sodium citrate, phosphate, tris(hydroxymethyl)aminomethane (TRIS) and/or 3-(N-morpholino)propanesulfonic acid (MOPS).

12. The formulation of claim 1, wherein said formulation is an aqueous formulation.

13. The formulation of claim 1, further comprising an enteric polymer.

14. The formulation of claim 13, wherein the enteric polymer is Eudragit® L 30 D-55.

15. The formulation of claim 1, further comprising a spray aid at a concentration suitable for spray drying.

16. The formulation of claim 14, wherein said spray aid comprises maltodextrin, lactose, gelatin, talc, triethylcitrate, or mixtures thereof.

17. The formulation of claim 15, wherein said spray aid comprises from about 1 wt % to about 15 wt % of said formulation.

18. A method for producing a powder comprising nanocoated viral particles, comprising drying the formulation of claim 1.

19. A method for producing a powder comprising enteric polymer coated nanocoated viral particles, comprising drying the formulation of claim 13.

20. The method of claim 18 or 19, wherein drying comprises freeze-drying or spray-drying.

21. A dried powder comprising nanocoated viral particles made from the formulation of claim 1 or 13.

22. The dried powder of claim 21, wherein the viral activity of the viral particles is at least about 70% of the viral activity prior to drying.

23. A dried powder comprising nanocoated viral particles produced by the method of claim 17 or 18.

24. The dried powder of claim 23, wherein the viral activity of the viral particles is at least about 70% of the viral activity prior to drying.

25. A pharmaceutical composition for oral administration comprising the dried powder of claim 21.

26. A pharmaceutical composition for oral administration comprising the dried powder of claim 22.

27. A pharmaceutical composition for oral or parenteral administration comprising the formulation of claim 1.

28. A pharmaceutical composition for oral administration comprising the formulation of claim 12.

29. A vaccine comprising the dried powder of claim 21.

30. A vaccine comprising the dried powder of claim 22.

31. A vaccine comprising the formulation of claim 1 or 13.

32. A replication competent vaccine for oral administration comprising the dried powder of claim 21.

33. A replication competent vaccine for oral administration comprising the dried powder of claim 23.

34. A replication competent vaccine for oral or parenteral administration comprising the formulation of claim 1.

35. A replication competent vaccine for oral administration comprising the formulation of claim 13.
